SEQUENCE LISTING (2 sequences)

SEQ ID NO: 1 - protein, 366 aa, Homo sapiens
MASMAAVLTWALALLSAFSATQARKGFWDYFSQTSGDKGRVEQIHQQKMAREPATLKDSLEQDLNNMNKFLEKLRPLSGSEAPRLPQDPVGMRRQLQEELEEVKARLQPYMAEAHELVGWNLEGLRQQLKPYTMDLMEQVALRVQELQEQLRVVGEDTKAQLLGGVDEAWALLQGLQSRVVHHTGRFKELFHPYAESLVSGIGRHVQELHRSVAPHAPASPARLSRCVQVLSRKLTLKAKALHARIQQNLDQLREELSRAFAGTGTEEGAGPDPQMLSEEVRQRLQAFRQDTYLQIAAFTRAIDQETEEVQQQLAPPPPGHSAFAPEFQQTDSGKVLSKLQARLDDLWEDITHSLHDQGHSHLGDP

SEQ ID NO: 2 - DNA, 1060 bp, Artificial sequence
  Description of artificial sequence  AA4RP Promoter
atgtcccttgcagccaagtctaattctaactaatacaaactaactctaaaaaatgaatatatattcaccaggggataggctatttcaagcagagggaagcctgtataaaggctcagggaatgctgtggttttatgtggcagcagatgagactggaaatgagtcaggatgagccacagtggaggatgaattaaatgggcaggagtgtggtagaaagacctgttggaggctatgaatgcaatcaaggtgacagacaactggtgcaatgatggtagtggaaatggaggagaggggattgattcaagatgcatttaggaccaagaatcgggagcttgtgaacgtgtgtatgagtactgtagacggagtgggtgtgtcatcagagaagatctgagcatttgggcttgctctcctcagaggccctgcgagtggagttcagcttttcctcatggggcaaatctyactttcgctccagttcctggggctcagagtccctggcccagatgcctcttgccatctcatcttcaccctgcctggcttcccttgcttgttccaggattgtttcataaagagggatgtggttggtctttaaccctatgaatgctggctgaggatgcctgcggaacctgtagtgaagctttcaggggctgctcgggttctggctggtaggtgaacactgtccatcttgccggctgggacacagtgactctgggtagttgtgtaagagaggggcccttggcagacaaacaggttcttctctgttggtgggccagccagcaggtcagtgggaaggttaaaggtcatggggtttgggagaaactgggtgaggagttcagccccatcccccgtaaagctcctgggaagcacttctctactggggcagcccctgataccagggcactcattaaccctctgggtgccagggaaagggcaggaggtgagtgctgggaggcagctgaggtcaacttcttttgaacttccacgtggtatttactcagagcaattggtgccagaggctcagggccctggagtataaagcagaatgtctgctctctgtgcccagac

(19) United States
(12) Patent Application Publication
(10) Pub. No.: US 2004/0198656 A1
(43) Pub. Date: Oct. 7, 2004

(54) Methods of screening molecules that are used to prevent cardiovascular diseases

(76) Inventors: Jamila Najib, Santes (FR); Zouher Majd, Marc en Baroeul (FR)

Correspondence Address: NIXON & VANDERHYE, PC, 1100 N GLEBE ROAD, 8TH FLOOR, ARLINGTON, VA, 22201-4714, US

(21) Appl. No.: 10/487,334

(22) PCT Filed: Sep. 6, 2002
(86) PCT No.: PCT/FR02/03040

(30) Foreign Application Priority Data
Sep. 7, 2001 (FR) 0111598

(51) Int. Cl. C12Q 1/68; G01N 33/53

(52) U.S. Cl. 514/12; 435/7.1; 435/6

(57) ABSTRACT

The invention relates to compositions and methods for screening molecules that are used for the prevention or treatment of metabolic syndrome, cardiovascular diseases and/or atherosclerosis. In particular, the invention relates to methods and kits for screening compounds based on determining the effect of test compounds on the activity of a novel protein which is similar to apolipoprotein AIV. The invention also relates to compositions and methods for reducing the concentration of triglycerides and/or reducing the concentration or the expression of apolipoprotein CIII (apo CIII) and/or reducing the concentration of VLDL and/or increasing the activity of LpL and/or of HL and/or for increasing reverse cholesterol transport.

The invention relates to compositions and methods for screening molecules useful for the prevention or treatment of metabolic syndrome, cardiovascular diseases and/or atherosclerosis. In particular, the invention relates to methods for screening compounds based on determining the effect of test compounds on the activity of a novel apolipoprotein AIV related protein (AA4RP). The invention also relates to compositions and methods for reducing the concentration of triglycerides and/or reducing the concentration or the expression of apolipoprotein CIII (apo CIII) and/or increasing the activity of hepatic lipase (HL) and lipoprotein lipase (LpL) and/or reducing the concentration of VLDL.

Metabolic syndrome and cardiovascular diseases have become diseases of most serious concern worldwide. Atherosclerosis, an accompanying disorder and a major cause of death (from myocardial infarction, cerebral embolism, etc.), is a multifactorial disease.

Since the discovery of the statins and the initiation of numerous therapeutic trials with these drugs, all of which have given positive results, attention has focused these past ten years on LDL (Low Density Lipoproteins). LDL cholesterol has been adopted as the main parameter by the health authorities because a direct correlation has been demonstrated between LDL levels and cardiovascular risk.

The published results of many epidemiological studies and a clinical trial (VAHIT) now show that, in addition to LDL, triglycerides and HDL (High Density Lipoproteins) play a key role in atherogenesis. HDL are anti-atherogenic lipoproteins; their concentration is inversely correlated with risk. The role of triglycerides, and particularly lipoproteins rich in triglycerides and/or containing apolipoprotein CIII (apo CIII), as an independent risk factor for atherosclerosis is no longer in question.

Apo CIII is a protein of 79 amino acids synthesized in liver and intestine [1]. It is a component of chylomicrons, VLDL (very low density lipoproteins) and HDL (high density lipoproteins) [2].

The concentration of apo CIII plays an important role in controlling the metabolism of plasma triglycerides and in determining plasma levels of potentially atherogenic triglyceride-rich lipoproteins [3], since it inhibits their lipolysis by blocking the activity of LpL.

Apo CIII concentration can be correlated with many pathophysiological conditions which underlie a predisposition to atherosclerosis and cardiovascular diseases.

It has also been shown that the apo CIII concentration in VLDL and LDL particles is a more specific measure of cardiovascular risk than plasma triglyceride levels [4].

The concentration of triglycerides is also controlled by the lipolytic action of LpL and HL. In vivo, the preferred substrates of LpL are large particles represented by the triglyceride-rich lipoproteins such as chylomicrons and VLDL, whereas HL more efficiently hydrolyzes smaller lipoproteins such as IDL and HDL [5, 6] (in particular HDL₂ [7]).

LpL and HL also play a role in reverse cholesterol transport through their action on HDL remodelling. Through its action on triglyceride-rich lipoproteins, LpL participates in the formation of pre-β-1-HDL, which are the first acceptors of cell-derived cholesterol [8-10].

Both HL and LpL take part in the formation of pre-β-HDL but the mechanism of action is different. HL hydrolyzes triglyceride-rich HDL₂ which results in the formation of small HDL of the pre-β-1-HDL type, thus participating in selective uptake of HDL cholesterol in the liver [11]. It has been shown that HL and SR-BI (hepatic receptor involved in HDL uptake and transfer of HDL cholesterol to liver) have a combined and not a parallel action in promoting cholesterol outflow [7], thereby demonstrating the essential role of the enzyme in the phenomenon of reverse cholesterol transport.

The discovery or development of molecules, methods or kits for modulating, and particularly for reducing the quantity of apo CIII is therefore a major advance in the therapeutic, diagnostic and/or experimental field.

The discovery or development of molecules, methods or kits for modulating, and particularly for reducing the quantity of triglycerides or VLDL or for regulating reverse cholesterol transport is therefore also a major advance in the therapeutic, diagnostic and/or experimental field.

The invention proposes an alternative solution to the therapeutic strategies of the prior art for the management of metabolic syndrome and cardiovascular diseases. In fact, the invention describes the functional characterization of a novel protein which represents a particularly advantageous target for research and development of active molecules. Furthermore, said target has the important advantage of being specific of the metabolic routes in question.

More particularly, the invention results from the identification of a novel protein regulating the metabolism of lipids and lipoproteins, particularly lipoproteins rich in triglycerides and in apolipoprotein CIII (apo CIII).

Said protein comprising 366 amino acids has the following primary structure (SEQ ID NO 1).

MASMAAVLTWALALLSAFSATQARKGFWDYFSQTSGDKGRVEQIHQQKMA
REPATLKDSLEQDLNNMNKFLEKLRPLSGSEAPRLPQDPVGMRRQLQEEL
EEVKARLQPYMAEAHELVGWNLEGLRQQLKPYTMDLMEQVALRVQELQEQ
LRVVGEDTKAQLLGGVDEAWALLQGLQSRVVHHTGRFKELFHPYAESLVS
GIGRHVQELHRSVAPHAPASPARLSRCVQVLSRKLTLKAKALHARIQQNL
DQLREELSRAFAGTGTEEGAGPDPQMLSEEVRQRLQAFRQDTYLQIAAFT
RAIDQETEEVQQQLAPPPPGHSAFAPEFQQTDSGKVLSKLQARLDDLWED
ITHSLHDQGHSHLGDP.

Said protein corresponds to the sequence of the protein RAP3 previously described as being potentially involved in liver regeneration (Genbank access number: AF202889.1: Van der Vliet H. N., Groenink M., Leegwater A. C. J. and Chamuleau R. A. F. M. Submitted (09-Nov.-1999) Experimental Hepatology, Academic Medical Center, Meibergdreef 9, Amsterdam 1105 AZ, Netherlands).

It also corresponds to the AA4RP protein (Apolipoprotein AIV-Related Protein) of patent application WO01/00803 A2 (SEQ ID NO 3 of said application) filed by the company Genset. Said AA4RP protein is a member of the family of apolipoproteins involved in lipid metabolism.

The invention now shows that the hereinabove protein named AA4RP is able to reduce the concentration of triglycerides and VLDL in vivo. The invention shows that said protein therefore allows the concentration of apo CIII, a marker of atherogenic lipids, to be reduced in vivo.

The invention therefore results from the discovery of the functional role of a novel protein, named AA4RP, in regulating the metabolism of lipoproteins, particularly those containing triglycerides and apo CIII.

The invention also describes the production of peptides derived from the primary structure of AA4RP, comprising one or more of the immunogenic domains of AA4RP.

The invention further describes the production, from immunogenic peptides, of antibodies which recognize AA4RP in human lipoproteins, or eventually in free form, and thereby enabling the assay thereof. The development of said anti-AA4RP antibody has made it possible to localize this apolipoprotein in HDL, confirming its very likely role in reverse cholesterol transport.

The invention thus describes a novel target and tools that may be used to develop novel active molecules for preventing or treating metabolic syndrome, cardiovascular diseases, and particularly atherosclerosis. The invention may also be used for detecting a predisposition or a risk of developing metabolic syndrome, cardiovascular pathologies and particularly atherosclerosis.

A first object of the invention is therefore based on the use of AA4RP for developing compounds which are active on cardiovascular or metabolic diseases.

Apolipoprotein AIV Related Protein (AA4RP)

Within the context of the invention, the term AA4RP (or AA4RP protein) denotes a polypeptide comprising the sequence SEQ ID NO 1 or any variants, derivatives or homologues. More particularly it is any natural variant of the sequence SEQ ID NO 1, resulting from polymorphism, splicing, mutations, and the like. Such natural variants may therefore comprise one or more mutations or substitutions, a deletion of one or more residues, etc. The term homologue furthermore designates the AA4RP polypeptides of other species, such as rodents, bovines, etc., for instance.

Generally speaking, the term AA4RP denotes a polypeptide having the sequence SEQ ID NO 1 or any derivative comprising one or more mutations, deletions, substitutions or additions of one or more amino acids. Preferably, the polypeptides are recognized by a polyclonal antibody produced from AA4RP having the sequence SEQ ID NO 1. Even more preferably, the analogs are polypeptides conserving at least one immunological or biological property of AA4RP. In particular, the biological properties are the regulation of triglyceride or apo CIII concentration, for example. Preferred variants comprise at least 80% of sequence SEQ ID NO 1, even more preferably at least 90% sequence identity with sequence SEQ ID NO 1. The degree of identity may be determined according to the CLUSTAL method, for example. Specific variants have a mutation or a substitution affecting at most five amino acids of the sequence SEQ ID NO 1.

Specific derivatives according to the invention are the hereinabove AA4RP fragments, particularly polypeptides comprising a part of the sequence SEQ ID NO 1. In this respect, an object of the invention is a polypeptide wherein it comprises a fragment of sequence SEQ ID NO 1 and wherein it contains at least one immunogenic domain of human AA4RP.

In fact, the application describes the production of polypeptide fragments of AA4RP, preferably containing an immunogenic portion of AA4RP, which are used for antibody production, assay tests, as competitors, and the like.

In particular, said polypeptides were developed by using algorithms to evaluate flexibility [12, 13], hydrophilicity [14, 15], antigenicity [16] and secondary structures [17, 18].

In this manner, the applicants have identified immunogenically or biologically interesting regions of the molecule. A specific example is a polypeptide corresponding to (or comprising) residues 20-114 of the sequence SEQ ID NO 1.

The polypeptide fragments according to the invention preferably contain fewer than 200 amino acids, more preferably fewer than 150 amino acids. They preferably contain at least one AA4RP epitope. Preferred polypeptides of the invention comprise fewer than 200 amino acids of the sequence SEQ ID NO 1 and contain at least residues 50-80 of the sequence SEQ ID NO 1.

Within the scope of the invention, the term “immunogenic fragment” denotes any portion of the polypeptide comprising an epitope, preferably a T or B epitope. Such fragment therefore advantageously comprises at least seven consecutive amino acids of the sequence SEQ ID NO 1, more preferably at least 10 consecutive amino acids of the sequence SEQ ID NO 1, even more preferably at least 15 consecutive amino acids of the sequence SEQ ID NO 1.

The inventive polypeptides may comprise heterologous residues added to the indicated amino acid sequence. Thus, an object of the invention is a polypeptide comprising all or part of the sequence SEQ ID NO 1, or a natural variant therof, and a heterologous component.

The heterologous component may correspond to amino acids, lipids, sugars, and the like. It may also be chemical, enzymatic, radiolabelled group(s) and the like. In particular, the heterologous component may be a marker, a screening agent, a stabilizing agent, an agent to enhance immunogenicity or facilitate production, a protective agent, an agent facilitating penetration of the peptide into cells, a toxin, or active compound, an antibody, etc.

The inventive polypeptides may be in soluble form, purified or complexed with a carrier molecule such as KLH or serum albumin, or any other inert molecule (for example synthetic), such as a bead for example. The polypeptides according to the invention are preferably free of contamination by naturally occurring blood proteins, particularly another apolipoprotein. In a specific embodiment, the polypeptides are coupled to a carrier molecule in particular for the production of antibodies. The coupling may be carried out by conventional methods [19, 20]. The polypeptides may also be conjugated or fused to any other polypeptidic or peptidic molecule such as for instance a peptide, polypeptide or a biologically active protein.

The polypeptides may be prepared by artificial synthesis according to conventional methods such as solid phase synthesis described in reference [21] and in the examples. Artificial synthesis is advantageous in so far as the resulting polypeptides are devoid of any contamination by naturally occurring products, particularly blood products. It is understood that the inventive polypeptides may be produced by any biological, enzymatic or genetic method, and particularly by expression of a corresponding nucleic acid in a suitable host cell. In this respect, another object of the invention lies in a nucleic acid molecule coding for a polypeptide such as defined hereinabove. Said nucleic acid may be a DNA or an RNA, preferably a cDNA, and may possibly comprise a promoter region. The nucleic acid may furthermore be cloned into a vector (for instance, a plasmid, cosmid, phage, virus, artificial chromosome, and the like).

Such nucleic acids may be used for the production of the inventive polypeptides in vitro or directly in vivo.

The inventive polypeptides may be used in screening tests, in titration tests or as standard for example for test calibration. They may also be used for regulating AA4RP activity in vitro or in vivo. They are especially interesting for the production of anti-AA4RP antibodies.

An object of the invention is based on a composition comprising a polypeptide such as defined hereinabove. A specific composition comprises a polypeptide containing all or part of AA4RP and a pharmaceutically acceptable vehicle. The vehicle may be a saline, isotonic, buffered solution, possibly combined with a stabilizing agent, emulsifier, and the like.

Antibodies

Another object of the invention is based on an antibody recognizing a polypeptide such as defined hereinabove. In an advantageous manner the antibody is specific of said polypeptide, that is to say, prepared by immunization with said polypeptide.

The antibody may be polyclonal or monoclonal. Moreover, the term antibody also designates any antibody fragments or derivatives, in particular fragments or derivatives of monoclonal or polyclonal antibodies conserving the same antigenic specificity. Such antibody fragments or derivatives comprise for example the fragments Fab, Fab'2, CDRs etc., humanized antibodies, polyfunctional antibodies, single chain antibodies (ScFv), etc. Said antibodies may be produced by conventional methods comprising immunizing an animal and recovering the serum (polyclonal) or spleen cells (for production of hybridomas by fusion with suitable cell lines).

Methods of production of polyclonal antibodies from various species such as mice, rodents, primates, horses, pigs, rabbits, fowl, etc. may be found in reference [19]. Briefly, the antigen is combined with an adjuvant, for instance Freund's adjuvant, and administered to an animal, usually by subcutaneous injection. Repeated injections may be given. Blood samples are collected and immunoglobulins or serum are isolated.

Methods for producing monoclonal antibodies may be found in particular in Harlow et al. [22] or in reference [23]. Briefly, said methods comprise immunizing an animal with an antigen followed by recovery of spleen cells which are then fused with immortalized cells such as myeloma cells. The resulting hybridomas produce monoclonal antibodies and may be selected by limit dilution to isolate individual clones. The antibodies may also be produced by selection of combinatorial immunoglobulin libraries, such as described for example in reference [24].

Fab or Fab'2 fragments may be produced by digestion with proteases according to conventional methods. Humanized antibodies may be prepared as described for instance in references [25, 26].

In a preferred embodiment, the invention relates to an antibody directed against an epitope comprised in residues 20-114 of the sequence SEQ ID NO 1.

According to another specific embodiment, the invention concerns any anti-AA4RP antibody, wherein it is obtained by immunizing a non-human mammal with an immunogenic peptide comprising a part of the sequence SEQ ID NO 1.

Such a polyclonal antibody was produced in rabbits against a synthetic peptide of 95 amino acids. Said antibody was used to demonstrate, by western blot, that AA4RP is localized mainly in HDL. In control mice, the subfraction recognized after two-dimensional immunoelectrophoresis was found in pre-βHDL which are the first acceptors of cell-derived cholesterol during reverse cholesterol transport.

The invention also concerns a method for producing an anti-AA4RP antibody comprising injecting a polypeptide such as defined hereinabove, particularly a polypeptide comprising all or part of the sequence SEQ ID NO 1, particularly residues 50 to 80, in a non-human mammal, then recovering the antibodies or antibody-producing cells.

The antibodies according to the invention may be coupled with heterologous components such as toxins, markers, drugs or other therapeutic agents. The coupling may be covalent or not, direct or by means of a coupling agent or spacer. The markers may be exemplified by radioactive markers, enzymes, fluorescent agents, magnetic particles, and the like. Examples of toxins are the diphtheria toxin, botulism toxin, ricin, and the like. The drugs or therapeutic agents are for instance lymphokines, antibiotics, antisense molecules, growth factors, and the like. Methods for carrying out the coupling of such heterologous regions are described for example in the American patent U.S. Pat. No. 4,277,149.

The antibodies of the invention have many uses particularly in therapeutics, diagnostics, prophylaxis and in the experimental field, for example for purifying antigens. In vitro, they may be used in particular as screening agent or for purifying antigen from various biological samples (blood samples such as plasma, or serum, or another biological fluid such as urine, interstitial fluid, etc.). They may also be used for detecting or quantifying the presence (or the quantity) of AA4RP in a sample taken from a subject or on a cell culture, typically a blood sample collected from a mammal, particularly a human being, as will be described hereinbelow.

Screening Tests

Another specific object of the invention is a method for selecting, identifying or characterizing compounds, wherein it comprises determining the ability of a test compound to modulate, in particular to increase, the activity of AA4RP.

Said method therefore allows the selection, identification or characterization of compounds able to reduce the level of circulating apolipoprotein CIII or of lipoparticles rich in apolipoprotein CIII and/or increase the activity of LpL and/or of HL and/or increase reverse cholesterol transport by determining the ability of a test compound to modulate (reduce or increase), in particular increase, the activity of AA4RP. More particularly, one defines compounds able to selectively increase AA4RP activity, that is to say, essentially without having a direct affect on the activity of another apolipoprotein. The inventive methods are especially useful for selecting, identifying or characterizing active compounds for treating atherosclerosis or cardiovascular diseases.

Within the context of the invention, the term “AA4RP activity” denotes in particular the synthesis of this protein (transcription, translation, etc.), its maturation, export or extracellular secretion, its interaction with a receptor, incorporation into particles (particularly HDL and VLDL), its degradation, and the like. The compound increasing AA4RP activity may therefore be an agent that increases the synthesis of AA4RP, an agent that increases AA4RP transport or secretion, a competitor of AA4RP, an agent that mimics the activity of AA4RP, and the like.

According to a first preferred embodiment, the method of the invention comprises determining the ability of a test compound to increase AA4RP synthesis, that is to say in particular the transcription or translation of its gene or RNA.

According to another preferred embodiment, the inventive method comprises determining the ability of a test compound to increase the concentration of AA4RP in HDL particles.

In another preferred embodiment, the inventive method comprises determining the ability of a test compound to increase the concentration of AA4RP in VLDL particles.

In a further preferred embodiment, the inventive method comprises determining the ability of a test compound to mimic the biological action of AA4RP. As illustrated in the examples, said biological action involves in particular reducing the concentrations of triglycerides or apo CIII.

According to another preferred embodiment, the inventive method comprises determining the ability of a test compound to mimic the biological action of AA4RP. As illustrated in the examples, said biological action involves in particular increasing the activity of LpL and/or HL. The inventive method may be implemented in different ways, in in vitro, cellular or acellular tests, or in vivo. These may be binding tests, functional tests, and the like.

A first test is based on measuring an interaction between a compound and AA4RP. In this embodiment, the method comprises contacting the test compound with AA4RP or a fragment thereof and measuring the binding of the test compound to AA4RP or to the fragment.

Said test may be carried out in vitro, in any suitable device (tube, dish, flask, etc.). It is possible to carry out with one of the partners immobilized, for example AA4RP (column, bead, support, glass, filter, membrane, etc.). Binding of the test compound may be demonstrated by any known method, particularly by electrophoresis, gel migration, immunochemistry, etc. In particular, binding may be demonstrated by carrying out the reaction in the presence of a labelled ligand of AA4RP and measuring the displacement of binding of the labelled ligand by the test compound. The labelled ligand may be an anti-AA4RP antibody or a fragment or derivative of such antibody, for example.

Another type of test according to the invention is based on the transcriptional activity of AA4RP. Said method comprises contacting the test compound with a nucleic acid comprising all or part of the promoter sequence of the AA4RP gene and determining binding of the test compound to the nucleic acid or modulation of nucleic acid activity by the test compound.

To carry out the binding test, it is possible to use a nucleic acid comprising all or part of the promoter sequence, and to determine in vitro the ability of a test compound to bind thereto. The part of the sequence preferably contains at least 10 consecutive nucleotides of the promoter sequence, even more preferably at least 20 consecutive nucleotides of the promoter sequence. Moreover, it is possible to test several fragments of the promoter sequence concurrently.

To carry out the transcriptional test, one advantageously uses a reporter system comprising all or part of the AA4RP gene promoter functionally linked to a reporter gene. In this respect, a preferred embodiment of the method comprises contacting the test compound with a cell comprising a reporter gene under the control of a transcriptional promoter comprising all or part of the promoter sequence of the AA4RP gene, and determining the effect of the test compound on the expression of the reporter gene. The sequence of the AA4RP gene promoter is represented in SEQ ID NO: 2.

Another embodiment of the inventive method comprises contacting the test compound with a cell expressing AA4RP or a fragment thereof and determining the effect of the test compound on the expression or secretion of AA4RP or the fragment by the cell.

The inventive methods may be carried out with different types of cells, promoter, reporter genes, and under different conditions, as described hereinbelow.

a) Host Cell

Some screening methods described by the invention provide for a step of contacting the test compound with host cells, under specific conditions allowing to determine the expression of a reporter genes in said cells, or the expression of AA4RP, or other steps of AA4RP synthesis, and thereby obtaining information concerning the effect of the test compound. Classically, the effect of the test compound is compared with the expression (of the reporter gene) or with the activity measured in the absence of said compound.

The cells used may be any cell that can be cultured in the laboratory. In a preferred embodiment, the cells are mammalian cells (hepatocytes, fibroblasts, endothelial cells, muscle cells, etc.). Even more preferably, said cells are human. They may be primary cultures or established cell lines. In another embodiment, it is also possible to use prokaryotic cells (bacteria), yeast cells (Saccharomyces, Kluyveromyces, etc.), plant cells, and the like.

b) Reporter System

In certain embodiments, the invention makes use of a reporter system comprising a reporter gene placed under the control of a specific promoter. Said construct, or any cassette or vector containing it, is introduced into host cells, that can be used for cellular tests.

In particular, said reporter gene may be any gene whose transcription or expression product can be detected or assayed in biological extracts. For example, it may be the gene coding for human AA4RP itself, or yet a gene coding for luciferase and more particularly for firefly or Renilla luciferase, for secreted alkaline phosphatase, galactosidase, lactamase, chloramphenicol acetyl transferase (CAT), human growth hormone (hGH), β-glucuronidase (Gluc) and green fluorescent protein (GFP) etc. It is understood that the term “gene” denotes in the broad sense any nucleic acid, particularly a cDNA, gDNA, synthetic DNA, an RNA, etc.

The reporter gene, whatever it may be, is placed under the control of a promoter comprising at least a part of the sequence of the promoter of the AA4RP gene such as defined hereinabove or a functional variant thereof. Said specific sequence may be present in one or more copies in the promoter (preferably 1 to 10 and even more preferably 1 to 6), upstream, downstream or internally, in the same orientation or in the opposite orientation. In a preferred embodiment of the invention, the reporter gene is placed under the control of a promoter which comprises the complete promoter sequence of the AA4RP gene, particularly human.

In this respect, an object of the invention also relates to a nucleic acid comprising a reporter gene under the control of a promoter comprising all or part of the promoter sequence of the AA4RP gene, particularly human. The sequence of the A4RP gene promoter is represented by SEQ ID NO: 2. The last four residues of the sequence correspond to the start of exon 1. Residues 1027 to 1032 correspond to the TATA box. The part of the promoter sequence advantageously comprises at least 10 consecutive nucleotides of said sequence, preferably at least 20, more preferably at least 30, even more preferably at least 50. The promoter may also comprise heterologous regions, from other genes or promoters, such as for example silencer or enhancer signals, sequences conferring a regulated or tissue specific character, etc. The promoter may be a hybrid promoter combining regions from other promoters, for example from the promoter of the herpes virus thymidine kinase (TK) gene, the CMV immediate early promoter, the PGK promoter, the SV40 promoter, etc.

The construct may be cloned into any suitable vector, such as a plasmid, cosmid, phage, virus, etc. The construct or vector may be introduced into a host cell by any classical method, such as electroporation, precipitation with calcium phosphate, liposomes, transfection agents, etc. The cells or their descendents may be grown in any suitable medium (DMEM, RPMI, etc.).

c) Contact

The test compounds may be contacted with the cells at different times, according to their effect(s), their concentration, the type of cells and technical considerations. Contact may be carried out on any suitable support and particularly on a plate, dish, in a tube or flask. Generally, contact is carried out in a multiwell plate which allows many and different tests to be conducted concurrently. Typical supports include microtiter plates and more particularly plates containing 96 or 384 wells (or more), which are easy to manipulate.

Depending on the support and nature of the test compound, variable quantities of cells may be used to carry out the herein-described methods. Classically, 10³ to 10⁶ cells are contacted with a type of test compound, in a suitable culture medium, and preferably between 10⁴ and 10⁵ cells. As an example, in a 96-well plate, 10⁵ cells may be incubated in each well with a desired quantity of test compound; in a 384-well plate, less than 10⁵ cells and typically between 1×10⁴ and 4×10⁴ cells are generally incubated in each well with the test compound.

The quantity (or concentration) of test compound may be adjusted by the user according to the type of compound (its toxicity, its ability to enter cells, etc.), the number of cells, the incubation time, etc. Generally, the cells are exposed to quantities of test compounds ranging from 1 nM to 1 mM. Of course other concentrations may be tested without deviating from the invention. Each compound may also be tested in parallel at different concentrations.

Différent adjuvants and/or vectors and/or products that facilitate penetration of the compounds inside cells such as liposomes, cationic lipids or polymers may also be used, where necessary.

Contact is typically maintained for several minutes to several hours, generally between 1 and 48 hours. In particular, when the test comprises the expression of a reporter gene, the cells and various reagents should preferably remain in contact long enough to enable de novo synthesis of the expression product of the reporter gene.

d) Measurement of Effect

The measurement or demonstration of an effect of the test compound may be carried out in different ways, according to the test used.

Methods for detection of binding in vitro have been described previously. For cellular tests involving detecting or determining the expression of a reporter system, several solutions are possible.

It may be a determination of transcriptional activity. To this end, total RNA is extracted from the cell cultures under the experimental conditions on the one hand and in a control situation on the other hand. The RNA is assayed (or used as probe) to analyze the variations in expression of the reporter gene(s).

It may also be a visualization or assay of the expression product of the reporter gene. Said visualization (or said assay) may be carried out by various methods according to the type of reporter gene used. The measurement may, for example, be an optical density or a fluorescent emission in the case where the gene coding for β-galactosidase or luciferase is used as reporter gene.

In a specific embodiment, the expression of the reporter gene is measured in terms of the hydrolysis of a substrate of the expression product of the reporter gene. For example, a number of substrates may be used to evaluate the expression of β-lactamase. In particular the substrate may be any product containing a β-lactam nucleus and whose hydrolysis can be controlled. Preferred substrates are those specific of β-lactamase (i.e., they are generally not hydrolyzed in mammalian cells in the absence of β-lactamase), those which are not toxic to mammalian cells and/or whose hydrolysis product can be easily measured, for instance by methods based on fluorescence, radioactivity, enzymatic activity or any other method of detection.

The expression product may also be assayed by immunological or immunoenzymatic methods, such as for example by means of a specific antibody. This system is especially suited to assaying AA4RP synthesized by a cell treated or not with a test compound, for example.

Generally, the presence of the reporter gene product (or the hydrolysis product of the substrate) can be determined by classical methods known to those skilled in the art (fluorescence, radioactivity, O. D., luminescence, FRET (see WO 0037077), SPA, biochips, immunological methods, etc.). Generally, one determines the activity of a test compound in a cell and this effect is compared with the activity observed in the absence of test compound or with a mean value determined in the absence of any test compound.

A second test allowing validation in animals of the selected compounds may also be carried out by determining the quantity of HDL expressed or by determining a significant variation in reverse cholesterol transport in cells treated with said compounds compared with untreated cells. It is also possible to measure plasma concentrations of triglycerides and/or apo CIII.

The compounds which may be identified by the inventive method may be compounds of different nature, structure and origin, particularly biological compounds, nuclear factors, cofactors, and the like, chemical, synthetic compounds and the like. They may also be libraries, particularly chemical libraries or protein, peptide or nucleic acid libraries, and the like.

Use of the Compounds

Considering the physiological functional properties of the target used, the compounds able to modulate, particularly to increase or mimic, AA4RP activity (eventually identified by the hereinabove methods) may be used for curative or preventive treatment of cardiovascular diseases or metabolic syndrome by reducing apolipoprotein CIII levels in the blood of a patient, in particular lipoparticles rich in apolipoprotein CIII, or by increasing the activity of LpL and/or HL or by increasing reverse cholesterol transport.

Said compounds may treat various pathologies, particularly cardiovascular pathologies, metabolic syndrome and in particular atherosclerosis.

In this respect, the invention generally relates to the use of a compound modulating, preferably increasing or mimicking, the activity of AA4RP for preparing a medicament for the curative or preventive treatment of metabolic or cardiovascular pathologies. More particularly, these are pathologies such as atherosclerosis (due to a dyslipoproteinemia or not), insulin resistance, type 2 diabetes, and the like.

Another object of the invention lies in the use of a compound increasing or mimicking AA4RP activity for preparing a medicament for reducing the levels of triglycerides and/or apo CIII in the blood, in particular in order to the level of lipoparticles rich in triglycerides or apo CIII.

Another object of the invention lies in the use of a compound increasing or mimicking AA4RP activity for preparing a medicament for increasing the activity of LpL and/or HL, in particular in order to reduce the level of triglycerides in lipoproteins.

Another object of the invention lies in the use of a compound increasing or mimicking AA4RP activity for preparing a medicament for regulating reverse cholesterol transport. In particular, said regulation takes place through an increase in the activity of LpL and/or HL and/or SR-BI.

In the context of the invention, the term “treatment” denotes preventive, curative, palliative treatment as well as management of patients (alleviating suffering, extending survival, improving quality of life, slowing disease progression, etc.). The treatment may also be conducted in combination with other agents or treatments, particularly addressing the late events of the disease or with other active substances. As noted hereinabove, the compounds used are preferably selective activators of AA4RP activity.

As indicated hereinabove, the compound used is preferably a compound which mimics the activity of AA4RP, stimulates AA4RP expression or secretion and/or increases the concentration of AA4RP in HDL particles which are known to play a protective role against atherosclerosis, mainly due to their ability to extract peripheral cell-derived cholesterol and promote its transfer to the liver where it is eliminated [27].

The invention also relates to methods of treating cardiovascular pathologies comprising administering to a subject a (selective) activator of AA4RP, particularly a compound which mimics the activity of AA4RP, stimulates AA4RP expression or secretion and/or increases the concentration of AA4RP in HDL particles. The administration may be by any route classically used in this type of therapeutic approach, such as in particular by the systemic or oral route, in particular, by injection, particularly intravenous, intradermal, subcutaneous, intraperitoneal, intramuscular, intra-arterial, etc.

Diagnostic Uses/Assays

The invention may also be used for the diagnosis, screening or detection of metabolic disorders.

In this respect, the invention therefore concerns a method for detecting a metabolic or cardiovascular disorder in a subject, or a predisposition to such disorder, comprising a step of determining, in a sample from the subject, the presence or the quantity of AA4RP or the corresponding RNA.

The invention also relates to a method for detecting a metabolic or cardiovascular disorder in a subject, or a predisposition to such disorder, comprising a step of determining, in a sample from the subject, the presence of an alteration in the sequence of AA4RP or its gene or RNA.

The invention may also be used for monitoring the efficacy of a therapeutic, vaccinal, dietetic treatment, etc., comprising assaying AA4RP in a sample from a subject. The assay may be carried out at different time points during the treatment.

The assay or detection of AA4RP may be carried out in an advantageous manner by means of an antibody such as described hereinabove. In this context, a quantitative assay in man may be advantageously developed on the IMMAGE® or ARRAY® immunochemistry system (Beckman Coulter, Villepinte, France) which enables a highly accurate assay of plasma proteins by kinetic immunonephelometry, competitive or not. It is understood that any other assay method, immunological or otherwise, for example RIA, ELISA, chromatography, Dot Blot, immunoturbidimetry, etc., may be used. Moreover, the assay may also be carried out on the RNAs encoding AA4RP. In this case, the assay may be done for example by hybridization with a nucleic probe complementary to all or part of the mRNA sequence.

In this respect, the invention also relates to a nucleic probe comprising all or part of the sequence coding for AA4RP and which is labelled. The probe is preferably a single stranded nucleic acid. It may be labelled by any known system, such as radioactivity, fluorescence, luminescence, enzyme, etc. The probe preferably comprises fewer than 500 nucleotides, more preferably fewer than 400 nucleotides. It may be immobilized on a support, for example a glass slide, silica, a nylon membrane, a bead, column, and the like.

The invention equally concerns kits for carrying out the methods of diagnosis, screening or detection of metabolic disorders, or for screening or selecting active molecules. Said kits advantageously comprise an antibody or a probe or a primer specific of AA4RP or a variant thereof and, advantageously, reagents for carrying out the detection or amplification or hybridization reactions. In an advantageous manner, the kits for selection of compounds may comprise a reporter system such as described hereinabove and reagents for detecting the reporter gene expression, or a nucleic acid comprising part of the AA4RP gene or promoter, for example.

The invention will be described in more detail in the following examples, which are given for purposes of illustration and not by way of limitation.

### LEGENDS OF FIGURES

FIG. 1: SDS PAGE control of anti-AA4RP antibody specificity and demonstration of AA4RP distribution in VLDL and HDL.

FIG. 2: Detection of AA4RP in plasma by two-dimensional electrophoresis and blotting.

FIG. 3: Effect of overexpression of AA4RP on triglyceride levels.

FIG. 4: Effect of overexpression of AA4RP on apo CIII levels in AA4RP transgenic animals and controls.

FIG. 5: Effect of overexpression of AA4RP on apo CIII levels in AA4RP “knock-out” homozygote and heterozygote animals.

FIG. 6: Triglyceride distribution in different lipoparticles in AA4RP transgenic mice and controls.

FIG. 7: HL and LpL enzymatic activity in mice transgenic for human AA4RP and in controls.

### EXAMPLES

### Example 1

### Peptide Sequence

1. Choice of Suitable Peptide Sequence

The following peptide fragment was synthesized. Said fragment was defined by using different algorithms to predict flexibility, hydrophilicity, antigenicity, and secondary structures. Said fragment containing 95 amino acids corresponds to residues 20 to 114 of sequence SEQ ID NO 1.

ATQARKGFWDYFSQTSGDKGRVEQIHQQKMAREPATLKDSLEQDLNNMNK
FLEKLRPLSGSEAPRLPQDPVGMRRQLQEELEEVKARLQPYMAEA

2. Peptide Synthesis

The peptide was synthesized by the solid phase synthesis method [21] on an ABI 431 A automatic synthesizer (Applied Biosystems Inc., California, USA) using a Boc/Bzl strategy on 0.5 mmol (0.57 mmol/g) MBHA resin. Each amino acid was coupled twice in the presence of dicyclohexylcarbodiimide/hydroxybenzotriazol without capping. Side chain protector groups were as follows: Arg(Ts), Asp(Ochex), Glu(Ochex), Lys(2-Cl-Z), His(Dnp), Ser(Bzl), Thr(Bzl), Met(O), Trp(formy) and Tyr(Br-Z).

The Dnp group on the histidine residue was eliminated from the peptide before cleavage from the support by treatment in the presence of 10% β-mercaptoethanol, 5% diisopropylethylamine in DCM medium for 2 hours then in NMP medium for 2 hours. The peptidyl resin was treated with 50% TFA in DCM medium for 20 minutes to eliminate the terminal amino acid Boc. The peptide was cleaved from the resin and simultaneously deprotected according to a slow and rapid HF procedure: the resin (1 g) was treated with anhydrous HF (2.5 ml) in the presence of p-cresol (0.75 g), p-thiocresol (0.25 g) and dimethylsulfide (0.5 ml) at 0° C.

Three hours later, the hydrogen fluoride and dimethylsulfide were eliminated by vacuum evaporation and the residual scavengers and secondary products were extracted with diethyl ether. The reaction vessels were reloaded with p-cresol (0.75 g), p-thiocresol (0.25 g) and 10 ml of anhydrous HF and the mixture was incubated at 0° C. for 1.5 hours. Hydrogen fluoride was eliminated by evaporation and the residue was mixed in the presence of diethyl ether. The residue was filtered, washed with diethyl ether and extracted with 200 ml of a 10% aqueous solution of acetic acid, then freeze-dried.

3. Mass Spectrometry

The molecular mass was determined on an ion electrospray mass spectrometer. The electrospray spectrum was obtained by using an API apparatus (Perkin-Elmer-Sciex) on a single quadrupole ion electrospray mass spectrometer, equipped with an ion spray (electrospray assisted by a nebulizer) (Sciex, Toronto, Canada).

4. Immunization

The peptide was emulsified in complete Freund's adjuvant and injected subcutaneously in rabbits at a dose of 0.5 mg per injection for the first two injections, followed by a booster dose of 0.25 mg of peptide in the same adjuvant every two weeks.

### Example 2

### Isolation of Rabbit Specific Anti-AA4RP Antibodies

Polyclonal antibodies were isolated by precipitation with 27% sodium sulfate then purified by affinity chromatography on activated Sepharose 4B gel (Pharmacia, Uppsala, Sweden), coupled with the AA4RP peptide residue 20 to 114 AA [28]. Proteins not retained on the antigenic gel were eliminated by washing with phosphate buffered saline (PBS: Phospate 50 mmol/L, pH 7.2, NaCl 150 mmol/L). Fractions not specifically bound on the AA4RP gel were eliminated with PBS 25 mmol/L. AA4RP-specific polyclonal IgG were eluted with 0.2 M glycine pH 2.8. The purified antibodies were immediately dialyzed against PBS 10 mmol/L then concentrated by ultrafiltration on an Amicon system (cutoff 100 kD) (Amicon, Dr. Bervely, MA, USA), assayed for protein content [29], then stored in 1 ml aliquots (1 mg) at −30° C.

1. Western Blot Analysis

1.1—Protocol:

Antibody purity and specificity were analyzed by western blot [30].

Human HDL, LDL and VLDL particles were subjected to denaturing SDS-PAGE electrophoresis (5 to 24%), then transferred to a nitrocellulose membrane and reacted with purified anti-human-AA4RP antibody. Immunoreactive proteins were visualized with a horseradish peroxidase-conjugated anti-IgG polyclonal antibody (Sanofi-Diagnostics Pasteur, Marnes-la-Coquette, France). The reaction was developed by chemiluminescence (Amersham, Pharmacia, Biotec).

1.2—Results

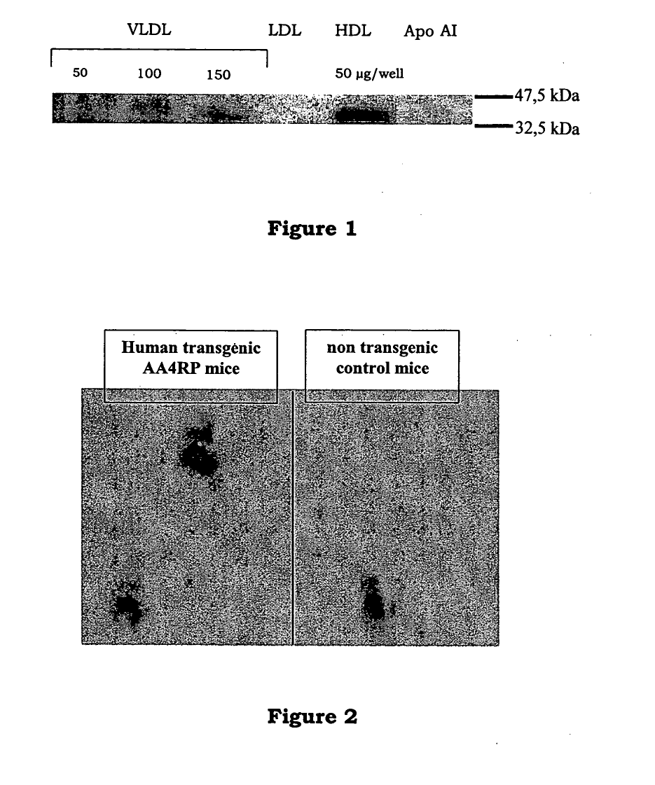

The results are presented in FIG. 1. It can be seen on this figure that the specific band revealed by the anti-AA4RP antibody is located between the 32.5 and 47.5 kDa molecular weight markers.

1.3—Interpretation:

The immunoblot results on the different human lipoproteins shows that AA4RP was localized in VLDL and HDL, primarily in HDL.

The presence of AA4RP in VLDL would explain the role of this apolipoprotein in metabolic regulation of these triglyceride-rich lipoproteins, and thus the modulation of the concentration of these atherogenic lipids.

The localization of AA4RP in HDL is undoubtedly related to the role of these particles in reverse cholesterol transport. In fact, the majority of apolipoproteins within HDL promote uptake of cell-derived cholesterol by HDL and transfer to the liver where it is eliminated.

2. Two-Dimensional Electrophoresis and Blotting

2.1—Protocol:

The following samples were studied:

plasma from control mice,

plasma from mice transgenic for human AA4RP.

Twenty microliters of each sample were loaded in 1.5 cm wells in a 0.75% agarose gel in 50 mmol barbital buffer pH 8.6 on Gelbond (FMC Bio-Products, New Jersey, USA). Electrophoresis was carried out in the first dimension until the bromophenol blue-stained albumin marker had migrated 7.5 cm. Electrophoresis in the second dimension was carried out in a 2 to 15% polyacrylamide gel gradient (15×15 cm). Samples (0.5×7 cm) were cut from the agarose gel, placed horizontally one after another in the polyacrylamide gel and covered with 1% agarose. Electrophoresis was carried out at 4° C. in 25 mM tris-(hydroxymethyl) aminomethane (Tris)-glycine buffer (pH 8.3) at 100 V for 19 hours. Gels were immunoblotted on nitrocellulose membranes (Sartorius 0.45 μm) in 25 mM tris-(hydroxymethyl) aminomethane (Tris)-glycine buffer (pH 8.3) using a semi-dry blotting system (Pharmacia, Umeå, Sweden). Membranes were incubated with anti-AA4RP polyclonal antibodies and those bound specifically to AA4RP were visualized with peroxidase-conjugated rabbit anti-IgG. The enzymatic reaction was developed by chemiluminescence (Amersham, Pharmacia, Biotec).

1.2—Results:

The results are shown in FIG. 2. They show that in transgenic and control mice, AA4RP was localized in pre-β-HDL, whereas the apolipoprotein was only present in α-HDL in the mice transgenic for human AA4RP.

1.3—Interpretation:

The presence of AA4RP in pre-β-HDL is a second argument in favor of the involvement of this apolipoprotein in cholesterol transfer from peripheral cells towards the liver. The presence of large amounts of AA4RP in α-HDL after transgenesis, while it was totally absent in this HDL subfraction in the control mice, is noteworthy. This different redistribution is presumably related to the maturation of HDL, where AA4RP would modulate the different factors involved in HDL metabolism, such as CETP (Cholesteryl Ester Transfer Protein), LCAT (Lecithin: Cholesterol AcylTransferase) or PLTP (Phospholipid Transfer Protein).

### Example 3

### Effect of Overexpression of AA4RP on Triglyceride Levels

1.—Protocol:

Samples from AA4RP transgenic mice or control mice maintained on a lipid-rich diet were diluted 1/3 or 1/5in physiological serum, then assayed for triglycerides against a calibration curve prepared with CFAS lipid calibrator, Ref. 759350 (Boehringer Mannheim GmbH, Germany). The calibration curve covered a concentration range of 16 to 500 μg/ml. 100 μl of each sample dilution or calibration point were deposited in each well of a 96-well titration plate. Next, 200 μl of triglyceride reagent Ref. 701912 (Boehringer Mannheim GmbH, Germany) were added to each well and the plate was incubated at 37° C. for 30 min. Optical densities (OD) were read on a spectrophotometer set at 492 nm. Triglyceride concentrations in each sample were calculated from a standard curve plotted as a linear equation y=ax+b, where y represents OD and x represents triglyceride concentration.

2—Results:

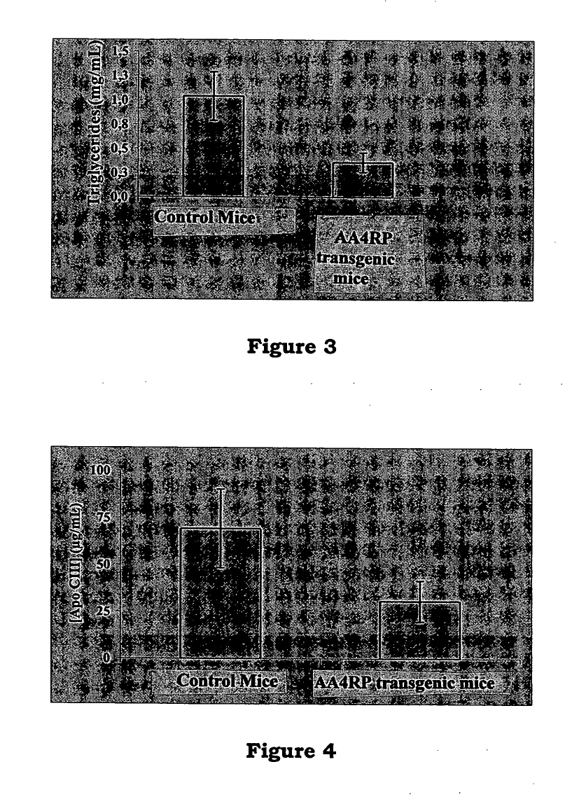

The results are presented in Table I below and in FIG. 3.

TABLE I

| | Control | AA4RP |
|---|---|---|
| | mice | transgenic mice |
| Number of animals | 13 | 21 |
| Triglycerides (mg/ml) | 1.039 | 0.361 |
| Standard deviation | 0.249 | 0.101 |
| (mg/ml) | | |

The results show that triglyceride levels were significantly lower in AA4RP transgenic mice (Student's t test p<0.001).

3—Interpretation:

The increase in triglyceride levels in control mice fed a lipid-rich diet was sharply reduced after transgenesis. This reduction is probably due to an acceleration of the catabolism of triglyceride-rich lipids such as chylomicrons and VLDL, following an increase in the activity of different catabolic effectors such as for example an increase in the activity of lipoprotein lipase (LpL), an increase in the cofactors of LpL activation or a decrease in factors such as apo CIII inhibiting said activity.

### Example 4

### Effect of Overexpression of AA4RP on apo CM Levels

1—Protocol:

Apo CIII in mice overexpressing human AA4RP was assayed by immuno-nephelometry on an ARRAY® immunochemistry system (Beckman Coulter, Villepinte, France). Pooled sera from normolipidic mice were used as standard; the apo CIII concentration therein was 25 μg/ml. A calibration curve from 4 μg/ml to 25 μg/ml was prepared by diluting the standard in 0.01 M potassium phosphate pH 7.2.

Plasma samples from mice overexpressing human AA4RP or not, maintained on a lipid-rich diet, and plasma samples from knock out homozygous (−/−), heterozygous (+/−) or wild type (+/+) mice were diluted 1/6 or 1/12 in the same conditions as the standard. Forty-two microliters of each calibration point and each sample from the different mice under study were incubated with 42 μl of mouse anti-apo CIII (in a reaction cell containing polyethylene glycol (PEG)). The apo CIII concentration in the sample is directly proportional to the light intensity reflected by the precipitate formed during the antigen-antibody reaction.

2—Results:

a—) Apo CIII in AA4RP Transgenic Mice and Control Mice (Lipid-Rich Diet)

The results are shown in Table II below and in FIG. 4.

TABLE II

| | Control | AA4RP |
|---|---|---|
| | mice | transgenic mice |
| Number of animals | 9 | 20 |
| Apo CIII (μg/ml) | 69.29 | 30.70 |
| Standard deviation | 20.90 | 10.88 |
| (μg/ml) | | |

The results show that the apo III level was approximately three times lower in AA4RP transgenic mice.

b—) Apo CIII in AA4RP Knock-out Homozygotes (−/−), Heterozygotes (+/−) and Wild-Type Mice (+/+):

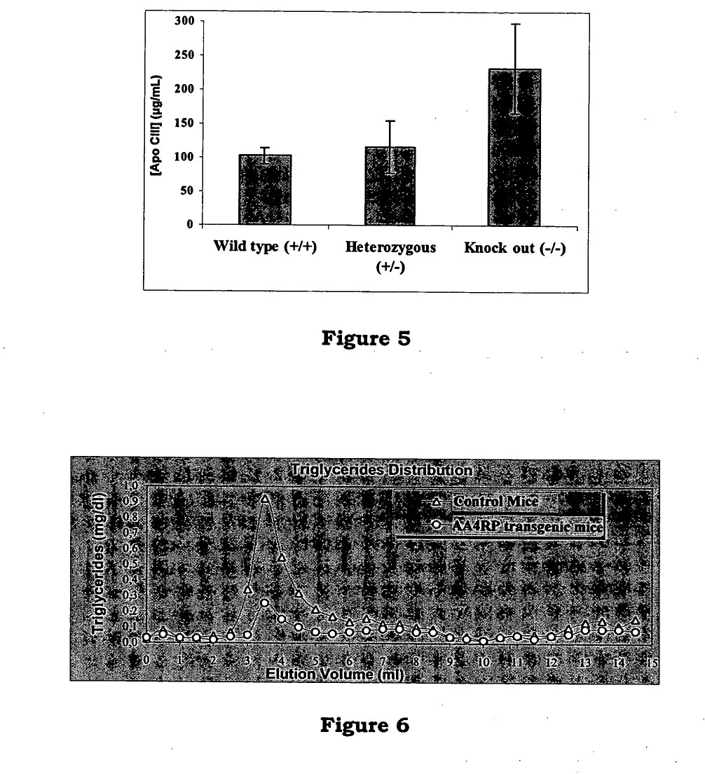

The results are presented in Table III below and in FIG. 5.

TABLE III

| | Wild type | KO mice | KO mice |
|---|---|---|---|
| | (+/+) | (+/−) | (−/−) |
| Number | 6 | 11 | 6 |
| Apo CIII (μg/ml) | 102.58 | 115.83 | 231.75 |
| Standard deviation | 11.94 | 38.24 | 66.33 |
| (μg/ml) | | | |

The results show that AA4RP knock-out mice had the highest levels of apo III.

3—Interpretation:

The observed reduction in apo CIII levels in AA4RP transgenic mice on a lipid-rich diet and the observed increase in apo CIII in AA4RP knock-out mice are in agreement with the results on triglyceride concentrations, thus confirming the positive role of AA4RP in the elimination of residual triglyceride-rich particles.

### Example 5

### Triglyceride Distribution in Different Lipoproteins

1—Protocol:

The distribution of triglycerides in VLDL, LDL and HDL was studied on 200 μl fractions of plasma from AA4RP transgenic mice or control mice maintained on a lipid-rich diet.

The different particles were fractionated on a Pharmacia Superose 6 column integrated in an AKTA FPLC system (Pharmacia Biotech, Umeå, Sweden). The different fractions were eluted with 0.01 M PBS, 0.1 M NaCl, pH 7.2 at a flow rate of 0.2 ml/min. The triglyceride concentration in each fraction was determined as described hereinabove.

2—Results:

The results are presented in FIG. 6.

3—Interpretation:

The accumulation of triglyceride-rich particles in control mice after a lipid-rich diet was not observed in the AA4RP transgenic mice, confirming the role of AA4RP in eliminating residual particles.

### Example 6

### LpL and HL Activity in Human AA4RP Transgenic Mice

1—Protocol:

The test was carried out on post-heparin plasma (collected 5 min after heparin injection) from transgenic mice and control mice with a FVB/N genetic background. Total lipase activity was determined by incubation with radiolabelled triolein and in the presence of a cofactor (apo CII). HL activity was determined after incubation of each plasma sample with triolein but in this case in the absence of apo CII and in the presence of 1 M NaCl to eliminate LpL activity. Lipase activity in each plasma sample was then calculated by determining the number of moles of free fatty acid. Finally, LpL activity was calculated by subtracting HL activity from total lipase activity.

2—Results:

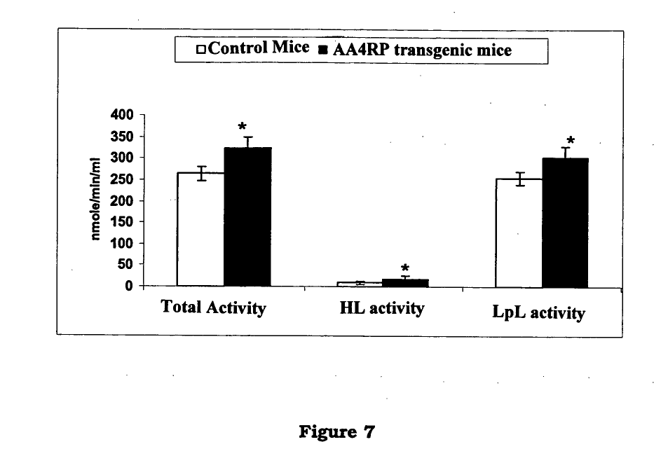

The results are presented in FIG. 7. They show that there was a significant increase in total lipase activity, HL activity and LpL activity in AA4RP transgenic mice as compared to controls.

3—Interpretation:

The increase in LpL and HL enzymatic activity in AA4RP transgenic mice is in agreement with the activity of AA4RP:

reduction of triglyceride and apo CIII concentrations (observed in examples 3, 4 and 5)

increase in cholesterol extraction and localization of AA4RP in HDL and pre-β-HDL so as to participate in their metabolism (example 2).

### BIBILIOGRAPHIC REFERENCES

1. Brewer, H. B., Shulman R., Herbert P., Roman R. and Werrly K., The complete amino acid sequence of alanine apolipoprotein from plasma very low density lipoprteins. J. Biol. Chem., 1974. 249: p. 4975-4984.

2. Lenich, C., Brecher P., Makrides S., Chobanian A. and Zannis V. I., Apolipoprotein gene expression in rabbit: abundance, size and distribution of apolipoprotein mRNA species in different tissues. J. Lip. Res., 1988. 29: p. 755-764.

3. Hoddis, H. N. and Mack W. J., Triglyceride-rich lipoproteins and the progression of coronary artery disease. Curr. Opin. Lipidol., 1995. 6: p. 209-214.

4. Sacks, F. M., Alaupovic P., Moye L. A., Cole T. G., Sussex B., Stampfer M. J., Pfeffer M. A. and Braunwald E., VLDL, apolipoproteins B, CIII, and E, and risk of recurrent coronary events in the Cholesterol and Recurrent Events (CARE) trial. Circulation, 2000. 102(16): p. 1886-92.

5. Kuusi, T., P. K. Kinnunen and E. A. Nikkila, Hepatic endothelial lipase antiserum influences rat plasma low and high density lipoproteins in vivo. FEBS Lett, 1979. 104(2): p. 384-8.

6. Jansen, H. et al., On the dual localization of lipoprotein lipase in rat heart. Studies with a modified perfusion technique. Biochem. Biophys. Res. Commun., 1980. 92(2): p. 411-6.

7. Shirai, K., R. L. Barnhart and R. L. Jackson, Hydrolysis of human plasma high density lipoprotein 2-phospholipids and triglycerides by hepatic lipase. Biochem. Biophys. Res. Commun., 1981. 100(2): p. 591-9.

8. Castro, G. R. and C. J. Fielding, Early incorporation of cell-derived cholesterol into pre-beta-migrating high-density lipoprotein. Biochemistry, 1988. 27(1): p. 25-9.

9. Huang, Y., A. von Eckardstein and G. Assmann, Cell-derived unesterified cholesterol cycles between different HDLs and LDL for its effective esterification in plasma. Arterioscler. Thromb., 1993. 13(3): p. 445-58.

10. Francone, O. L., A. Gurakar and C. Fielding, Distribution and functions of lecithin:cholesterol acyltransferase and cholesteryl ester transfer protein in plasma lipoproteins. Evidence for a functional unit containing these activities together with apolipoproteins A-βI and D that catalyzes the esterification and transfer of cell-derived cholesterol. J. Biol. Chem., 1989. 264(12): p. 7066-72.

11. Barrans, A. et al., Hepatic lipase induces the formation of pre-beta 1 high density lipoprotein (HDL) from triacylglycerol-rich HDL2. A study comparing liver perfusion to in vitro incubation with lipases. J. Biol. Chem., 1994. 269(15): p. 11572-7.

12. Westhof, E. et al., Correlation between segmental mobility and the location of antigenic determinants in proteins. Nature, 1984. 311(5982): p. 123-6.

13. Karplus, P. A. and G. E. Schulz, Prediction of chain flexibility in proteins. Naturwiss-enschaften, 1985. 72: p. 212-216.

14. Hoop, T. and K. Woods, A computer program for predicting protein antigenic determinants. Molecular Immunology, 1983. 20: p. 483-489.

15. Kyte, J. and R. F. Doolittle, A simple method for displaying the hydropathic character of a protein. J. Mol. Biol., 1982. 157(1): p. 105-32.

16. Jameson, B. A. and H. Wolf, The antigenic index: a novel algorithm for predicting antigenic determinants. Comput. Appl. Biosci., 1988. 4: p. 181-186.

17. Chou, P. Y. and G. D. Fasman, Prediction of the secondary structure of proteins from their amino acid sequence. Adv. Enzymol. Relat. Areas Mol. Biol., 1978. 47: p. 45-148.

18. Garnier, J., D. J. Osguthorpe and B. Robson, Analysis of the accuracy and implications of simple methods for predicting the secondary structure of globular proteins. J. Mol. Biol., 1978. 120(1): p. 97-120.

19. Vaitukaitis, J. et al., A method for producing specific antisera with small doses of immunogen. J. Clin. Endocrinol. Metab., 1971. 33(6): p. 988-91.

20. Bassiri, R. M., Dvorak J. and Utiger R. D., Thyrotropin-releasing hormone, in Methods of Hormone Radioimmunoassay, B. M. In: Jaffe and Behrman H. R. (Eds). 1979, New York Academic Press: New York. p.: 46.

21. Merrifield, R. B., Solid phase peptide synthesis. The synthesis of a tetrapeptide. J. Am. Chem. Soc, 1963. 85: p. 2149-2154.

22. Harlow, E. A. L., D., ed. Antibodies a Laboratory Manual. 1988, Cold Spring Harbor Laboratory Publications: New York. pp: 139-243.

23. Kohler, G. and C. Milstein, Continuous cultures of fused cells secreting antibody of predefined specificity. Nature, 1975. 256(5517): p. 495-7.

24. Ward, E. S. et al., Binding activities of a repertoire of single immunoglobulin variable domains secreted from Escherichia coli. Nature, 1989. 341(6242): p. 544-6.

25. Jones, P. T., Dear P. H., Foote J., Neuberger M. S. and Winter G., Replacing the complementarity-determining regions in a human antibody with those from a mouse. Nature, 1986. 321: p. 522-525.

26. Riechmann, L., Clark M., Waldmann H. and Winter G., Monoclonal antibody therapeutic trials in seven patients with T-cell lymphoma. Nature, 1988. 332: p. 323-327.

27. Fruchart, J. C. and P. Duriez, High density lipoproteins and coronary heart disease. Future prospects in gene therapy. Biochimie, 1998. 80(2): p. 167-72.

28. Axen, R., J. Porath and S. Emback, Chemical coupling of peptides and proteins to polysaccharides by means of cyanogen halides. Nature, 1967. 214(95): p. 1302-4.

29. Lowry O. H., R. J. Farr A. L., and Randall R. J., Protein measurement with folin phenol reagent. J. Biol. Chem., 1951. 193: p. 265-275.

30. Towbin, H., T. Staehelin and J. Gordon, Electrophoretic transfer of proteins from polyacrylamide gels to nitrocellulose sheets: procedure and some applications. Proc. Natl. Acad. Sci. USA, 1979. 76(9): p. 4350-4.

1-10. canceled.

11. A method for selecting, identifying or characterizing compounds for reducing the level of circulating apolipoprotein CIII or lipoparticles rich in apolipoprotein CIII and/or increasing the activity of lipoprotein lipase and/or of hepatic lipase and/or increasing reverse cholesterol transport in a mammal, comprising determining the ability of a test compound to modulate the activity of the AA4RP protein.

12. A method according to claim 11, wherein it comprises determining in vitro or ex vivo the ability of a test compound to modulate the synthesis of the AA4RP protein.

13. A method according to claim 11, wherein one determines the ability of the test compound to increase the activity of the AA4RP protein.

14. A method according to claim 11, wherein it comprises contacting in vitro or ex vivo a test compound with a cell comprising a reporter gene under the control of a transcriptional promoter containing all or part of the sequence of the AA4RP gene promoter, and determining the ability of said test compound to modulate the expression of the reporter gene.

15. A method according to claim 11, wherein it comprises determining the ability of a test compound to increase the incorporation of the AA4RP protein into HDL or VLDL particles, in particular in HDL particles.

16. A method for the curative or preventive treatment of cardiovascular pathologies or metabolic syndrome by reducing apolipoprotein CIII levels in the blood of a patient, in particular lipoproteins rich in apolipoprotein CIII, or by increasing the activity of LpL and/or of HL or by increasing reverse cholesterol transport, by administering to a subject in need of such treatment a compound modulating the activity of the AA4RP protein.

17. A method according to claim 16, wherein the compound modulating the activity of the AA4RP protein is a compound increasing or mimicking the activity of the AA4RP protein.

18. A method according to claim 16, for the curative or preventive treatment of atherosclerosis.

19. A method according to claim 16, wherein the compound modulating the activity of the AA4RP protein stimulates or inhibits its expression or its secretion.

20. A method according to claim 16, wherein the compound modulating the activity of the AA4RP protein increases or decreases the concentration of the AA4RP protein in HDL particles.

21. Polypeptide fragments of AA4RP containing fewer than 200 amino acids, more preferably fewer than 150 amino acids of the sequence SEQ ID NO 1.

22. Polypeptide fragments of AA4RP according to claim 21, containing at least residues 50-80 of the sequence SEQ ID NO 1.

23. Polypeptide fragments of AA4RP according to claim 21, comprising residues 20-114 of the sequence SEQ ID NO 1.

24. Polypeptide fragments of AA4RP according to claim 21, comprising at least seven consecutive amino acids of the sequence SEQ ID NO 1, more preferably at least 10 consecutive amino acids of the sequence SEQ ID NO 1, even more preferably at least 15 consecutive amino acids of the sequence SEQ ID NO 1.

25. A composition comprising a polypeptide, fragment of AA4RP, containing fewer than 200 amino acids, more preferably fewer than 150 amino acids of the sequence SEQ ID NO 1.

26. A pharmaceutical composition, comprising a polypeptide, fragment of AA4RP, containing fewer than 200 amino acids, more preferably fewer than 150 amino acids of the sequence SEQ ID NO 1, and a pharmaceutically acceptable vehicle.

27. A pharmaceutical composition according to claim 26, for regulating, in particular increasing or mimicking, AA4RP activity in vivo.

28. A pharmaceutical composition according to claim 26, for the curative or preventive treatment of cardiovascular pathologies or metabolic syndrome by reducing apolipoprotein CIII levels in the blood of a patient, in particular lipoproteins rich in apolipoprotein CIII, or by increasing the activity of LpL and/or of HL or by increasing reverse cholesterol transport.
